(19) United States
(12) Patent Application Publication
(10) Pub. No.: US 2011/0184195 A1
(43) Pub. Date: Jul. 28, 2011

(54) PROCESS FOR THE HYDROGENOLYSIS OF FURFURYL DERIVATIVES

(76) Inventors: Jean-Paul Lange, Amsterdam (NL); Jeroen Van Buijtenen, Amsterdam (NL)

(21) Appl. No.: 12/999,445

(22) PCT Filed: Jun. 24, 2009
(86) PCT No.: PCT/EP09/57899

§ 371 Date: Apr. 14, 2011

(30) Foreign Application Priority Data
Jun. 24, 2008 (EP) 08158900.4

(51) Int. Cl. C07D 307/36

(52) U.S. Cl. 549/506

(57) ABSTRACT

The present invention relates to a process for the hydrogenolysis of a furfuryl derivative to 2-methylfuran derivative, comprising: (a) contacting under liquid phase conditions a solution of the furfuryl derivative in a solvent having a boiling point above the boiling point of furfuryl derivative with hydrogen in the presence of a catalyst comprising a hydrogenation compound to form a 2-methylfuran derivative and water, at a temperature and pressure suitable to maintain the furfuryl derivative in the solvent in the liquid phase, and (b) continuously distilling the 2-methylfuran derivative from the reaction mixture.

### FIELD OF THE INVENTION

The invention provides a process for the hydrogenolysis of furfuryl derivatives, such as furfural and 5-hydroxymethylfurfural, into the equivalent methylfuran derivatives, such as 2-methylfuran and 2,5-dimethylfuran, respectively. The invention further relates to the conversion of carbohydrates derived for instance from cellulose to methylfuran derivatives.

### BACKGROUND OF THE INVENTION

It is known that furfuryl derivatives, such as furfural and 5-hydroxymethylfurfural can be converted into the corresponding furan derivatives, such as 2-methylfuran and 2,5-dimethylfuran, respectively via the following hydrogenolysis reactions:

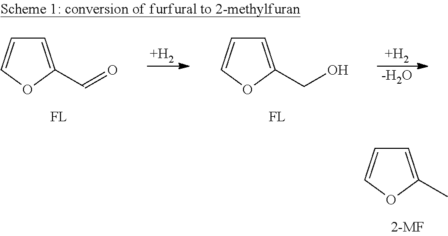

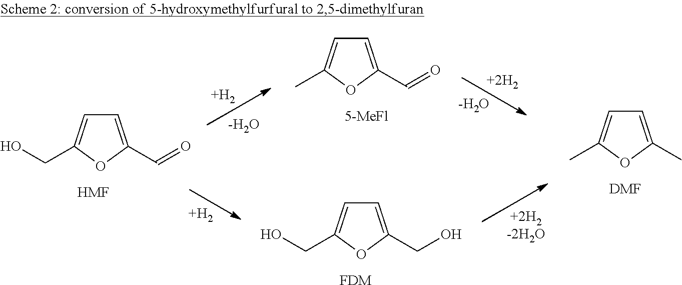

The furan derivatives are known as derivatives of pentose and hexose sugars, as set out for instance in WO 2007/146636. In Stonkus V. V. et al. “Characteristics of the catalytic hydrogenation of 5-methylfurfural” Chemistry of Heterocyclic Compounds 11 (1990), p-1214-1218, for example, a gas-phase conversion of furfural into 2-methylfuran using an industrial copper-chromite catalyst promoted by alkaline earth metal salts at conversion temperatures between 200 and 300° C. is disclosed.

Also the gas-phase conversion of 5-methylfurfural into 2,5-dimethylfuran using different catalysts is disclosed herein, using an industrial copper-chromite catalyst promoted by alkaline earth metal salts at conversion temperatures between 200 and 300° C.; using a Pd/C catalyst at conversion temperatures between 110 and 200° C.; and using a Pd/alumina catalyst at conversion temperatures between 100 and 200° C.

An article by G. Roberti et al., “Reazioni con catalizzatori in sospensione. Idrogenazione del furfurolo a silvano”, Annali di Chimica, 45 (1955), p. 193-204, discloses the reduction of furfural to 2-methylfuran in the liquid phase. The furfural is injected into a CuCr₂O₄ catalyst suspension in mineral oil at 245-250° C. and 2 atm. of hydrogen pressure, and a product stream containing unreacted furfural, 2-methylfuran and water are condensed from the gas phase. A disadvantage of the process is that the separation of unreacted furfural, 2-methylfuran and water from the ternary mixture is difficult.

An article by S. Morikawa, “Reduction of 5-Hydroxymethylfurfural”, Noguchi Kenkyu Jiho, 23 (1980), p. 39-44, discloses the conversion of 5-hydroxymethylfurfural by hydrogenation using palladium on active carbon as catalyst and Lewis acid as promoter using cyclohexane as hydrogen donor in toluene solvent under reflux. The conversion temperature is 80° C.

In a thesis titled “Hydrothermal conversion of carbohydrates and related compounds” by G. C. A. Luijkx, Delftse Universitaire Pers, Delft, 1994, p. 93-104, is disclosed the production of 2,5-dimethylfuran via hydrogenolysis of 5-hydroxymethylfurfural in simple organic solvents, or in water. Specifically, the hydrogenolysis of 5-hydroxymethylfurfural in 1-propanol using a Pd on alumina catalyst with and without the addition of a small amount of hydrogen chloride is disclosed, as well as the hydrogenolysis of 5-hydroxymethylfurfural in 1-propanol, in 2-propanol, in 1,4-dioxane, in water and in water-toluene using a Pd on active carbon as catalyst. Hydrogen chloride was added in the experiments in water and water-toluene. All experiments were carried out at 60° C. using hydrogen.

In WO 2007/146636, the acid-catalysed dehydration of fructose into 5-hydroxymethylfurfural in a reactor containing a bi-phasic reaction medium is disclosed, wherein the dehydration is carried out in an aqueous reaction solution and the 5-hydroxymethylfurfural formed is extracted into a substantially immiscible organic extraction solution comprising a solvent. Solvents selected from 1-butanol, dichloromethane, methylisobutylketone, and 2-butanol are mentioned as particularly preferred extraction solvents. After extraction, the 5-hydroxymethylfurfural is subjected to hydrogenolysis for conversion into 2,5-dimethylfuran in the presence of the extraction solvent and using a carbon-supported copper-ruthenium catalyst or a copper-chromite catalyst. The exemplified hydrogenolysis reactions are carried out in the liquid phase with 1-butanol or 1-hexanol as solvent or in the vapour phase with 1-butanol as solvent, all at 493 K (220° C.). Finally, 2,5-dimethylfuran as obtained and water were separated from the solvent and the intermediates by distillation. As set out on page 16, lines 9-11 of WO-A-2007/146636, it is proposed to recycle the thus obtained stream comprising solvent and intermediates to the hydrogenolysis reactor.

The above-described processes, in particular when performing the reaction in liquid phase, suffer from several drawbacks. Firstly, the catalyst activity rapidly declines within hours. Secondly, as the processes require a highly diluted feed, this results in a relatively low throughput, and expensive equipments for feed-effluent heat exchange and product recovery. Moreover, the report shows that the selectivity is low as evidenced by the modest reported carbon balance (in the range of from 70-860). Yet further, the disclosed processes require the vaporisation of large amounts of solvents to isolate the methylfuran derivatives after the hydrogenolysis reaction, which requires a high energy use.

Luijkx reports the formation of unspecified co-products labelled “others” than generally exceeds that of the desired DMF product, while Dumesic reports the formation of ring-hydrogenation products as well as a modest carbon balance of 80-92 C %. The modest C-balances of either process suggest the formation of oligomeric material that is prone to fouling of the catalyst. The gradual decay of catalyst activity in the above reactions has also been confirmed by the applicants.

### SUMMARY

Applicants have now found that by removing 2-methylfuran derivatives from the reaction mixture by carrying out the hydrogenolysis reaction under stripping conditions, some or most of the drawbacks reported above are overcome.

Furthermore, when preparing products for use as fuel components, crude mixtures comprising both furfural and HMF may be co-processed. This permits the use of feedstocks containing hexose as well as pentose sugars, such as those derived from fermentation of cellulose.

Accordingly, the subject invention relates to a process for the hydrogenolysis of a furfuryl derivative to 2-methylfuran derivative, comprising:

(a) contacting under liquid phase conditions a solution of the furfuryl derivative in a solvent having a boiling point above the boiling point of furfuryl derivative with hydrogen in the presence of a catalyst comprising a hydrogenation compound to form a 2-methylfuran derivative and water, at a temperature and pressure suitable to maintain the furfuryl derivative in the solvent in the liquid phase, and
(b) continuously distilling the 2-methylfuran derivative from the reaction mixture.

### DESCRIPTION OF THE DRAWINGS

FIG. 1 discloses a process for the preparation of 2-methylfuran derivatives from cellulose.

FIG. 2 discloses a preferred embodiment for the work-up section of this process.

### DETAILED INVENTION

In step (a), a feed comprising a furfuryl derivative is fed to a reactor. The feed or the reactor, or both contain an inert high-boiling solvent, a suitable hydrogenation catalyst and, optionally, a co-catalyst such as Broensted or Lewis acid. The reactor is heated up, or maintained at a temperature of 100-200° C. and continuously stripped by passing a H₂ containing gas stream over or through the reaction mixture at moderate pressure, i.e. less than or equal to 10 bar (atm) in such a way as to continuously withdraw at least part of the reaction products, i.e. the light-boiling MF and/or DMF, and co-produced water. The temperature, pressure and feed rates of (hydroxymethyl) furfural and H₂ are chosen such as to maintain the effective liquid-phase concentration of furfuryl alcohol moieties at sufficiently low level, preferably below 10 wt %, more preferably below 1 wt %, as to minimise to formation of oligomeric by-products that would otherwise foul the catalyst; and preferably to maintain the effective liquid-phase concentration of the (di)methylfuran product low enough, preferably below 10 wt %, more preferably below 1 wt %, as to minimise its degradation to e.g. tetrahydrofuran moieties. The optimal set of operating conditions obviously depends on catalyst parameters as well such as catalyst loading, activity and selectivity.

The present invention concerns the conversion of a furfuryl derivative to 2-methylfuran derivative. Within the present specification, the term furfuryl derivative relates to a compound having the following structure:

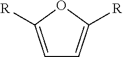

wherein R is independently selected from the group consisting of hydrogen, C₁-C₆-alkyl, hydroxy-C₁-C₆-alkyl, acyl-C₁-C₆ alkyl, C₁-C₆-alkylcarbonyl-C₁-C₆-alkyl and carboxy-C₁-C₆-alkyl, provided that at least one group R comprises a carbonyl structure, such as a ketone or an aldehyde, preferably a formyl substituent.

Preferably, the furfuryl derivative relates to furfural and 5-hydroxymethylfurfural and mixtures thereof, while the term 2-methylfuran derivative relates to 2-methylfuran and 2,5-dimethylfuran, respectively.

In step (a) the temperature is preferably in the range of from 80 to 200° C., and wherein the pressure is at most 10 bar (absolute). The liquid solvent preferably has a boiling point of at least 80° C., more preferably at least 100° C. The liquid solvent preferably has a boiling point of at most 400° C., more preferably at most 300° C. In the case of organic solvents, the liquid solvent preferably has a boiling point in the in the range of from 80 to 400° C., more preferably of from 100 to 300° C. Preferably, the liquid solvent is an organic solvent that is a liquid at ambient temperature and pressure, and more preferably a liquid under the hydrogenolysis conditions.

More preferably, the solvent is selected from gamma valerolactone, alkyl pivalate esters, 1^{ary} and 2^{ary} butanol and heavier alcohols such as tetrahydrofufuryl alcohol, aromatic solvents, such as toluene and xylenes, dibutyl ether and heavier ethers, or mixtures thereof. Higher alcohols within the present specification refers to alcohols heavier than 1^{ary} and 2^{ary} butanol, i.e. alcohols having a higher molecular weight.

The process may be applied to a wide range of product concentrations. Preferably, the liquid phase comprises in the range of from 0.1 to 20 wt % of the furfuryl derivative.

Although the process may be performed in batch reactions, it preferably is done in a continuous process scheme. Accordingly, a liquid feedstock comprising both the furfuryl and the liquid solvent is continuously supplied to the liquid phase.

The main product from hydrogenation of HMF in step (a) is 2,5-dimethyl furan, while FL is converted to 2-methylfuran. Selective hydrogenation of HMF or FL proceeds through reduction of an aldehyde group and further elimination of 2 water molecules. Further hydrogenation of 2-MF or 2,5-dimethyl furan or may lead to saturation of the aromatic ring, or even ring opening. These products are less desirable due to their lower energy content as fuel component, and the higher hydrogen consumption, which negatively will impact the process economics.

Therefore the suitable catalyst should be selected to facilitate selective hydrogenation of the furfuryl compound. Preferably, the hydrogenating compound in step (a) preferably is palladium, copper, ruthenium, or combinations thereof.

More preferably, the hydrogenating compound is copper or palladium, cooper being the most preferred. In this step, the furfuryl derivative is contacted with the hydrogen in the presence of an acidic catalytic function. Advantageously, the acidic catalytic function may be incorporated in the catalyst comprising palladium or copper. However, the acidic catalytic function may also be a liquid acid, preferably hydrochloric acid, sulphuric acid, phosphoric acid or p-TSA.

In step (b), the distillation is preferably performed under a continuous stripping gas flow. This may be performed by bubbling the stripping gas through the reaction mixture, for instance by using a bubble flow column or a similar reactor to allow the gas to flow through the reaction mixture, thereby entrailing light components, or fixed bed reactors, for instance in the shape of a distillation column, whereby the catalyst is packed on the liquid trays of the column, or with dedicated low-pressure drop catalyst packings.

Preferably, the furfuryl derivative and hydrogen are reacted in a reaction zone of a reactive distillation column. More preferably, the stripping gas comprising the hydrogen is continuously supplied to the liquid phase.

The H₂-containing stream may consist of pure H₂ or preferably of a diluted H₂ stream, such as H₂/CH₄. The reaction solvent should preferably meet at least one, preferably more than one the following requirements:
(1) have an atmospheric boiling point that is significantly higher than that of the 2-methylfuran derivative, preferably above 100° C., more preferably above 150° C.,
(2) be inert under the reaction conditions and should, therefore, contain no C═C, C═O, C═N bond,
(3) have an intermediate polarity to e.g. expressed as LogP between −1 and 2.

The gaseous effluent stream consists of H₂, the optional gas-diluents, methyl- and dimethylfuran, water and, optionally, other volatile components present in the feed, or produced by the reaction. This gaseous stream is advantageously worked-up by condensing the furfuryl derivatives and water from the gas stream, allowing natural separation of the condensate into an aqueous phase and the desired furan-rich phase, and washing residual furan moieties from the gas stream with the liquid feed or with the reaction solvent that is subsequently recycled to the reaction vessel.

A person skilled in the art will realise that, compared to the set-up known so far, the present set-up requires less equipment by combining the reaction and product separation in a single vessel, utilising the heat of reaction to heat-up the feed to reaction temperature and vaporise the reaction products, (di)methylfuran and water, and avoiding the need for extensive heating-cooling cycles of large solvent. A person skilled in the art will also realise that this set-up avoids the occurrence of hot spots that would otherwise favour the formation of undesirable by-products.

5-hydroxymethylfurfural (further referred to as HMF herein) as a preferred furfuryl derivative can be obtained from conversion of various sugars, most easily from conversion of fructose. However, fructose is a rather expensive starting material, making the processes not commercially attractive.

Accordingly, it would be desirable to be able to use an abundant and cheap glucose or cellulose, the latter comprising glucose building blocks as feedstock for HMF production.

This may be achieved by the enzymatic hydrolysis (fermentation) of cellulose, resulting in an aqueous solution of glucose as a product, which could be further treated to produce HMF. A further option is chemical hydrolysis, such as the treatment with the dilute solution of strong acid (e.g. sulfuric acid). However, the latter remains in the solution after the biomass liquefaction process.

However, until now, there was no known commercial process that permits to produces HMF on a commercial scale from cellulose, while glucose is solely known for a small scale HMF production. Acid catalyzed dehydration of C-6 sugars leads to the formation of HMF under release of 3 water molecules. This reaction is however fraught by a number of side reactions. For instance levulinic and formic acids are formed as by-products of the acid catalyzed HMF re-hydratation. HMF is also known to polymerize or to react with sugar intermediates to form solid humins. This usually results in significantly lower yields compared to those obtained from fructose. In order to be converted to HMF, glucose must undergo isomerisation to fructose, which proceeds at high temperatures or under base conditions. In this rather slow equilibrium reaction, around 20% of fructose is formed which in turn is then available for further reactions, alongside glucose and mannose. The fructose formed can then be dehydrated to HMF, catalysed under acidic conditions. Accordingly, both an acid and a base catalyst are required to allow formation of HMF from glucose.

Re-hydration of HMF to levulinic and formic acid is a further side reaction that affects the efficiency of the process. Being an acid catalyzed reaction, formation of these products enhances degradation of HMF in an autocatalysis fashion especially in the aqueous solutions. Therefore attempts have been made to increase the productivity of HMF by using non-aqueous systems.

Applicants have carried out a number of experiments to confirm the possibility for an HMF production from different sugar-based and cellulosic feedstock. In these experiments, different solvents, catalyst and temperatures were used to identify the most promising combination that could be applied on a large-scale process.

Firstly, the conversion of glucose to fructose and on to HMF was investigated. Experiments with glucose in water as solvent showed that, depending on the acidity of the catalyst glucose was isomerised to fructose or mannose. The amount of observed isomers was at most 40 mol %. More acidic catalysts gave mannose as a dominant isomerisation product, while in the experiments with less acidic catalysts, fructose was observed as main isomerisation product. This suggests that weak acids or acid systems such as YbCl₃, Formic Acid and Pyridine/H₃PO₄ catalyse the isomerisation of glucose to mannose and fructose but are less effective in catalyzing the subsequent dehydration of fructose to HMF.

In contrast, strong acids such as H₂SO₄ were highly effective dehydration catalysts, but also converted fructose rapidly. Preheating the glucose/water solution decreased drastically the measured amount of glucose implying intensive oligomerization to humins or oligo-sugars. Experiments with weakly acidic catalysts gave relatively good selectivity to HMF, with little production of levulinic and formic acid. However, the HMF yields observed did not exceed 20% in water. Addition of organic solvent to water increased the yield of HMF as compared to solely aqueous systems, although the observed reaction rates remained in the same range. Different organic solvents in combination with water, whether miscible or immiscible, gave almost identical results, including a reduced formation of levulinic acid and formic acid. In particular the butanol/water system formed a single phase at reaction temperature.

Non-aqueous systems with DMSO and Methyl Immidazolium Chloride prevented consecutive hydration of HMF to levulinic and formic acid, and yields of HMF were improved compared the aqueous system. The reaction rates in the experiments with DMSO were higher. C-2 acids such as acetic acid (AA), glycolic acid (GA) and glyoxilic acid (GOA) formed in large amount in the presence of weak acid catalyst. Their yields are also increasing at higher reaction temperatures. Typically they are produced in yields of 5-15% at for instance 170° C.

Elevated temperature was found to improve the reaction rates and yields of almost all the products, but did not improve HMF selectivity.

Accordingly, the present process further relates to the preparation of a 2-methylfuran derivative, comprising: (a1) dehydration of a pentose and/or hexose-containing feed to obtain a liquid feedstock comprising the furfuryl derivative and water, and (a2) supplying the liquid feedstock to step (a) of the process according to anyone of the preceding claims.

The feed stream may consist of purified furfural and/or hydroxymethyl furfural. Alternatively, it may consist of a crude dilute stream that stems from a previous reaction or recovery process. This latter case in particularly beneficial in the case of a feed containing hydroxymethyl furfural, which is otherwise difficult to purify.

When cellulose is used as feed instead of glucose, a stronger acid, and longer contact times for the hydrolysis are required. The insolubility of cellulose in water makes the hydrolysis a rather slow step, which determines the overall rate of reaction to produce HMF. Cellulose hydrolysis thus preferably is an independent pretreatment step, followed by furfuryl derivative production. This is preferably done under addition of valeric acid (VA) to the cellulose, since this improved the overall yields of useful products HMF and furfural.

Employing either DMSO and Methyl Immidazolium Chloride as solvents allowed HMF production (about 10%) directly from cellulose. Preferably this is further improved by performing the reaction on higher temperatures with very fast heating to the reaction temperature. The cellulose employed may also be lingo-cellulose due to its wide availability.

The present process has the further advantage that a pentose and/or hexose-containing feed may be employed, without having to separate and purify the products. Preferably, the pentose and/or hexose-containing feed is obtained from a cellulosic starting material.

In the process according to the present invention, the liquid feedstock may advantageously be obtained by extracting the furfuryl derivative from a stream comprising the furfuryl derivative by a solvent.

Suitable solvents for the furfural extraction include those which show significant affinity with furfural and preferably not with water.

Suitable solvents may be selected based on their Hansen solubility parameters. The Hansen solubility parameters as described in “Hansen Solubility Parameters, a users handbook by C. M. Hansen, ISBN 0-8493-1525-5, 2000 CRC Press, split the Hildebrand parameter into three different molecular interactions; a dispersive interaction δd (non permanent dipole—dipole interaction), a polar interaction δp (permanent dipole) and a hydrogen bonding interaction δh:

δHSB2=(δd)2+(δp)2+(δh)2[MPa]

The parameters themselves are given in [Mpa]^{0.5}. When components dissolve in each other the difference in solubility parameters should be small (“like dissolves in a like” concept). Mathematically this can be expressed in as δs:

δs=√[((δdi−δdj)²+(δpi−δpj)²+(δhi−δhj)²]

wherein δdi=dispersive interaction parameter component i; δdj=dispersive interaction parameter component j;
δbpi=polar interaction parameter component i; δpj=polar interaction parameter component j; δhi=hydrogen bonding interaction parameter component i; δhj=hydrogen bonding interaction parameter component j;
The solvent are selected by setting component i is furfural and component j is solvent molecule. Where δs is smaller than a certain value the components i and j dissolve in each other. Preferably, solvents are chosen wherein δs is below <10 [Mpa]^{0.5}, more preferably below 4 [Mpa]^{0.5}. These include N-Acetyl Pyrrolidone, Acrylonitrile, Butadienedioxide, 3-Butenenitrile, 2,3-Butylene Carbonate, Gamma-Butyrolactone, Epsilon-Caprolactone, 1-Chloro-1-Nitroethane, 4-Chloro-2-Nitrotoluene, Chloroacetonitrile, 2-Chlorocyclohexanone, Chloronitomethane, 3-Chloropropionaldehyde, Chloropropionitrile, Crotonaldehyde, Cyclobutanone, Cyclopentanone, Cyclopropylnitrile, Di-n-Proprl Sulfoxide, Diphenyl Sulfone, 2,3-Dibromoprene, Dichloromethyl Methyl Ether, 2,3-Dichloronitrobenzene, Diethyl Sulphate, Diketene, Dimethyl Methyl Phosphonate, Epsilon-Caprolactam, Ethanesulfonychloride, Ethyl Carbylamine, Ethyl Thiocyantae, Ethylene Glycol Sulphite, Ethynlidene Acetone, Fumaronitrile, Malononitrile, Methacrylonitrile, 4-Methoxy Benzonitrile, 3-Methoxypropionitrile, Methyl Isopropenyl Ketone, Methyl Nitrate, Methyl Sulfolane, Methy Thiocyanate, Methyl Vinyl Ketone, N-Methyl-2-Pyrrolidone, Nitroethane, Nitroethylene, 1-Nitropropane, 2-Nitropropane, Phenyl Acetonitrile, Propionitrile, Propylene Carbonate, Propynonitrile, Succinonitrile, Sulfolane, 2,2,6,6-Tetrachlorocyclohexanone, Tigaldehyde, 3,3,3-Trichloro Propene, 1,1,2-Trichloro Propene, 1,2,3-Trichloro Propene, Tricresyl Phosphate, and mixtures thereof.

Alternatively, as a liquid solvent, an ionic liquid may be employed. Although the use such a solvent led to high conversion and yields, these solvents are rather expensive and the formation of water in the hydrogenolysis reaction also reduced the effectiveness over time due to solvation. Such an ionic liquid does not have measurable boiling point, and therefore is particularly suitable for the reaction, with the drawbacks set out above. Methyl Immidazolium Chloride (HMIMCl) as solvent and catalyst gave very good selectivity for the formation of HMF over levulinic acid as side product, as already described in Moreau, C., A. Finiels, and L. Vanoye, Journal of Molecular Catalysis A: Chemical 2006. 253: p. 165-169.

Since separation and purification of HMF have proven highly difficult, it would be desirable to convert the formed HMF directly to its hydrogenolysis product, and to remove the latter. Therefore, preferably, the solvent employed to extract the furfuryl derivative from the hexose- or pentose sugar containing feed is the same as applied in the hydrogenolysis.

### DETAILED DESCRIPTION OF THE DRAWINGS

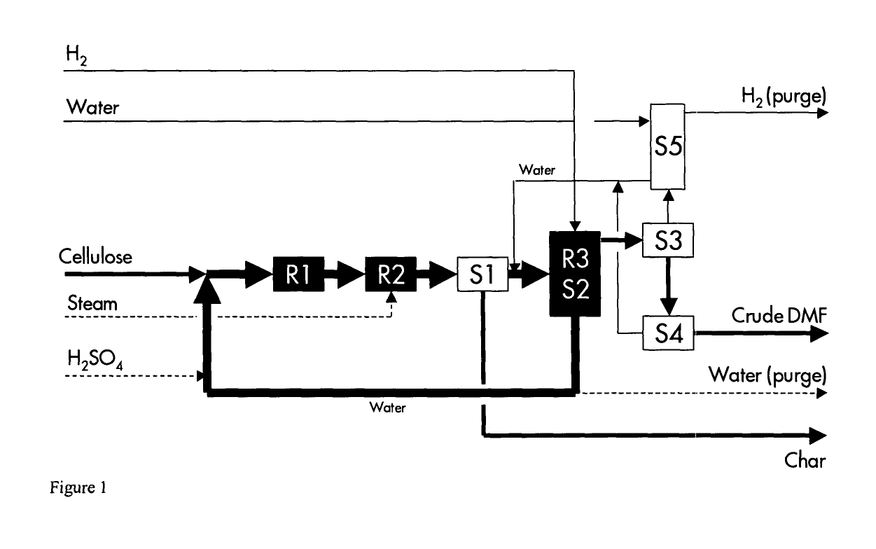

In FIG. 1, cellulose is mixed with recycle water and fed to the digester R1, where the slurry is partly hydrolysed at 120° C., and subsequently fed to the hydrolysis reactor R2, where the carbohydrates are fully hydrolysed and dehydrated to products (mainly HMF) and char at 150-180° C. The aqueous stream is then liberated from suspended char in the filter S1 and fed to the hydrogenation reactive distillation unit R3/S2 together with fresh H₂, where the HMF is hydrogenated to DMF at 80-150° C. and an azeotropic mixture of DMF and water, alongside other volatile organic components such as formic acid, acetic acid and MF are simultaneously stripped off the aqueous stream by excess H₂. The water-rich bottom stream of R3/S2 is recycled to the R1 after addition of make-up H₂SO₄.

The azeotropic vapour is recovered by condensing the most of the heavier component out of the H₂-rich gas in S3 and liberated from water via spontaneous liquid-liquid separation in the decanter S4 to produce a crude DMF product stream. The H₂-rich stream is cleaned from organic vapour (mainly DMF) by means of a water-wash in S5 and purged off the plant. The aqueous phase that exits S4 is combined with the wash water of S5 and sent back to the reactive distillation unit R3/S2.

FIG. 2 shows an alternative preferred embodiment of the work-up section. Herein, the reactive distillation unit R3/S2 is operated as two separated units, i.e. a hydrogenation reactor R3 and a subsequent distillation unit S2.

The invention will further be illustrated by the following, non-binding examples:

### EXPERIMENTS

The following experiments illustrate that a high yield to 2-methyl-furan can be achieved by the process line-up according to the invention. It is further illustrated that this may be achieved using different hydrogenation catalysts (such as the exemplified CuCrBa and Pd/Titania catalysts). The selectivity to total useful gasoline components was even higher in both examples.

The experiments were run using a 300 mL autoclave that was equipped with an electrical heating jacket, a gas-dispersing stirrer, two baskets placed symmetrically as baffles to hold the catalyst granules, an HPLC liquid pump and a mass flow controller for continuous supply of furfural and H₂, a gas outlet equipped with pressure release valve to control the pressure of the vessel while continuously releasing the stripping H₂ gas and two cold traps placed in series to condense the liquid product, one operating at −10° C. and the −80° C.

### Examples 1 and 2

Catalyst and solvent as set out in Table 1 were placed in the above-described autoclave. The substrate was then added using the HPLC pump, while a flow of gaseous hydrogen was employed to strip of the light products obtained in examples 1 and 2. Comparative examples 1 and 2 did not employ, or only a very low hydrogen stream. Components that can be formed under hydrogenolysis conditions are as follows: Furfural (FL) is rapidly hydrogenated to furfuryl alcohol (FAlc), while hydrogenolysis of FAlc affords MF (2-methyl-furan). Undesired ring hydrogenation of FAlc affords tetrahydrofurfuryl alcohol (THFAlc), while ring-hydrogenation of MF gives 2-methyl-tetrathydrofuran (MTHF) which can also be used as fuel component. Conversion of FL into furfuryl alcohol is almost instantaneous under these conditions and therefore both FL and FAlc are grouped together as “unconverted substrate” for the purpose of calculating conversions and yields. Yields refer to the conversion of total amount of reactant added during the experiment into products (which were found in the reactor after 5 h, or, where applicable, were found in the products collected from distillation). Not all by-products could be identified at this point in time or were too heavy to be analysed by GC, and hence were marked as unknown and missing products, respectively. In example 2 and comparative example 2, these by-products are likely to include products formed by reaction of MF or THFAlc with 2-ethylhexanol. The sum of the yields of MF and MTHF, THFAlc and unknown products amounted to 100%. The following catalyst were employed: Example 1 and comparative example 1 employed Catalyst 1, a commercial CuCrBa catalyst (Cu-1152, available from the Engelhard corporation).

Example 2 and comparative Example 2: Catalyst 2, catalyst prepared by incipient wetness impregnation of Palladium on TiO₂ catalyst comprising 3% Pd on TiO₂.

The examples below show that more MF and less unknown/missing products are produced when the product is continuously removed by stripping from the reaction mixture, as compared to a reaction where the product remains in the reaction mixture and is separated off after the reaction. Furthermore, similar experiments show that 2,5-DMF and generally useful fuel components are formed in higher yields from HMF applying the process according to the invention.

TABLE 1

| | Example 1 | Comp. 1 | Example 2 | Comp. 2 |
|---|---|---|---|---|
| Stripping conditions | Yes | No | Yes | No |
| Catalyst | 1 | 1 | 2 | 2 |
| Amount catalyst [g] | 13 | 12 | 10 | 10 |
| Solvent** | GVL | GVL | 2-EHA | 2-EHA |
| Solvent at start of | 166 | 192 | 170 | 171 |
| reaction [g] | | | | |
| Temperature [° C.] | 170 | 170 | 120 | 120 |
| Pressure | 7.5 bar | 8 bar | 2.0 bar | 2.5 bar |
| H₂ Flow (nL/h) | 45 | 1.5 | 35 | <1* |
| Furfural feed | 26/2.5 | 32/3.0 | 28/2.7 | 31/2.9 |
| (mmol/h)/(g/h) | | | | |
| Cumulative furfural | 12.3 | 15.2 | 13.3 | 15.0 |
| added (g) | | | | |
| total feed ([wt/wt], | 1:2 | furfural | 1:4 | furfural |
| including solvent to | furfural/ | | furfural/ | |
| compensate for | GVL** | | 2-EHA** | |
| distillation) | | | | |
| WHSV [g_{furfural}/g_{cat}/h] | 0.2 | 0.25 | 0.3 | 0.3 |
| H₂:furfural feed | 71 | 2 | 51 | n.a. |
| (molar ratio) | | | | |
| Mass balance | 97% | 95% | 99% | 99% |
| Conversion of FL + FAlc | 63.0% | 35.9% | 94.3% | 79.6% |
| Yields | | | | |
| to MF | 31.3% | 16.6% | 19.5% | 4.2% |
| to MTHF | 9.7% | 2.4% | 26.0% | 2.7% |
| to THFAlc | 12.3% | 3.8% | 8.8% | 0.8% |
| To unknown/missing | 9.8% | 13.1% | 40.0% | 71.9% |

*occasional supply of H₂ to compensate for pressure drop due to hydrogen consumption;
**Gamma-valerolactone is abbreviated as GVL, and 2-ethyl-hexanol as 2-EHA.

1. A process for the hydrogenolysis of a furfuryl derivative to 2-methylfuran derivative, comprising:
(a) contacting under liquid phase conditions a solution of the furfuryl derivative in a solvent having a boiling point above the boiling point of the furfuryl derivative with hydrogen in the presence of a catalyst comprising a hydrogenation compound to form a 2-methylfuran derivative and water, and
(b) continuously distilling the 2-methylfuran derivative from the reaction mixture.

2. The process of claim 1 wherein the distillation is performed under a continuous stripping gas flow.

3. The process of claim 1, wherein the temperature is in the range of from 80 to 200° C., and wherein the pressure is at most 10 bar (absolute).

4. The process of claim 1 wherein the liquid solvent has a boiling point in the range of from 80 to 400° C.

5. The process of claim 1 wherein the liquid solvent is an organic solvent that is liquid at ambient temperature and pressure.

6. The process of claim 1 wherein the solvent is selected from gamma valerolactone, alkyl pivalate esters, 1^{ary} butanol, 2^{ary} butanol, or higher alcohols aromatic solvents, dibutyl ether and ethers of higher alkanes, and/or mixtures thereof.

7. The process of claim 1 wherein the furfural and hydrogen are reacted in a reaction zone of a reactive distillation column.

8. The process of claim 1 wherein a stripping gas comprising hydrogen is continuously supplied to the liquid phase.

9. The process of claim 1 wherein the liquid phase comprises in the range of from 0.1 to 20 wt % of the furfuryl derivative.

10. The process of claim 1 wherein a liquid feedstock comprising both the furfuryl and the liquid solvent is continuously supplied to the liquid phase.

11. The process of claim 1 further comprising:
(a1) dehydration of a pentose and/or hexose-containing feed to obtain a liquid feedstock comprising the furfuryl derivative and water, and
(a2) supplying the liquid feedstock to step (a) of the process.

12. The process of claim 11 wherein the liquid feedstock is obtained by extracting the furfural derivative from a stream comprising furfural with a solvent.

13. The process of claim 11 wherein the hydrogenating compound is palladium and wherein the furfuryl derivative is contacted with the hydrogen in the presence of an acidic catalytic function.

14. The process of claim 13 wherein an acidic catalytic function is incorporated in the catalyst comprising palladium.

15. The process of claim 13, wherein the acidic catalytic function is a liquid acid.

16. The process of claim 15 where in the liquid acid is selected from hydrochloric acid, sulphuric acid, phosphoric acid or p-TSA.

17. The process of claim 2 wherein the liquid solvent has a boiling point in the range of 100 to 300° C.

18. The process of claim 6 wherein the solvent is selected from tetrahydrofufuryl alcohol, toluene, xylenes, and/or mixtures thereof.
